(19) United States
(12) Patent Application Publication
(10) Pub. No.: US 2024/0145026 A1
(43) Pub. Date: May 2, 2024

(54) PROTEIN TRANSFORMATION METHOD BASED ON AMINO ACID KNOWLEDGE GRAPH AND ACTIVE LEARNING

(71) Applicant: ZJU-HANGZHOU GLOBAL SCIENTIFIC AND TECHNOLOGICAL INNOVATION CENTER, HANGZHOU, ZHEJIANG PROVINCE (CN)

(72) Inventors: QIANG ZHANG, HANGZHOU, ZHEJIANG PROVINCE (CN); MING QIN, HANGZHOU, ZHEJIANG PROVINCE (CN); ZHICHEN GONG, HANGZHOU, ZHEJIANG PROVINCE (CN); HUAJUN CHEN, HANGZHOU, ZHEJIANG PROVINCE (CN)

(21) Appl. No.: 18/278,170

(22) PCT Filed: Oct. 21, 2022
(86) PCT No.: PCT/CN2022/126697

§ 371 Date: Aug. 21, 2023

(30) Foreign Application Priority Data
Feb. 9, 2022 (CN) 202210121706.8

(51) Int. Cl. G16B 15/00; G16B 35/00; G16B 40/20

(52) U.S. Cl. CPC G16B 15/00; G16B 35/00; G16B 40/20

(57) ABSTRACT

The present invention discloses a protein transformation method based on an amino acid knowledge graph and active learning, including: building an amino acid knowledge graph based on biochemical attributes of amino acids; enhancing protein data in combination with the amino acid knowledge graph to obtain enhanced protein data, and performing representation learning to obtain first enhanced protein representations; performing representation learning on the protein data or the protein data and the amino acid knowledge graph by using a pre-trained protein model to obtain second enhanced protein representations; synthesizing the first enhanced protein representations and the second enhanced protein representations to obtain enhanced protein representations; taking the enhanced protein representations as samples, and through active learning, screening out representative samples from the samples, manually annotating protein properties, and training a protein property prediction model by using the manually annotated representative samples; and performing protein transformation by using the protein property prediction model. Therefore, rapid and accurate protein transformation can be implemented.

### TECHNICAL FIELD

The present invention belongs to the technical field of representation learning for proteins, and specifically relates to a protein transformation method based on an amino acid knowledge graph and active learning.

### BACKGROUND TECHNOLOGY

A knowledge graph aims to objectively describe entities in the objective world and their relationships in the form of graphs. An amino acid knowledge graph describes the properties and attributes of various amino acids. Embedding representations of proteins can be obtained based on the amino acid knowledge graph in combination with deep learning to accelerate the prediction of various downstream protein properties, the scientific research, and the biological industrialization. Conventional supervised learning requires a large number of annotated data to learn the representations of proteins in a low-dimensional space. Protein tags need to be obtained by expensive equipment and trained experts in a time-consuming and resource-consuming manner, so that the introduction of priori knowledge of the knowledge graph can make up for the disadvantage of being prone to underfitting of models due to insufficient annotated data on the one hand, and can assist in active learning to achieve an objective of training models with higher prediction accuracy with less annotated data and lower cost on the other hand.

Active learning is usually applied to scenarios with rich unannotated data and high annotation costs. Through active learning, the most representative sample set can be selected from a large number of unannotated samples and is subjected to sample annotation, and then the annotated samples are applied to model training, which can achieve the higher training efficiency and save the manpower, material resources and time costs. As an important kernel method using a probabilistic generative model, a Fisher Kernel has been widely used in scenarios such as protein homology detection and speech recognition. The learnable Fisher Kernel learns by a principle that the gradients of samples of the same class are as close as possible and the gradients of samples of different classes are as different as possible.

The protein property prediction driven by machine learning, namely, the avoidance of a conventional idea of putting forward a hypothesis to conduct experimental verification, requires experimenters to have a large amount of knowledge in the art and to be trained for years. Meanwhile, the experimental verification requires a large amount of staffs to participate in, and many experiments also require expensive instruments, resulting in time and labor consumption and high costs. Through machine learning, a machine learning model is trained using experimental data accumulated in past experiments. In the following exploration, a target protein sequence with good performance can be found with few or no manual experiments and is in accordance with a protein function expected by the experimenters.

The embedding representations of the proteins serve as basis data for training protein property models. In general, the embedding representations of the proteins may represent protein sequences by open-source pre-trained models such as MSA-transformer and ESM, or mainly through expert knowledge such as Geogiev encoding. However, these methods have the following obvious disadvantages: firstly, the direct use of the pre-trained models can only focus on potential semantic knowledge of global protein sequences, so that the representation effect of each specific protein sample may not be good and rule knowledge obtained by human experts for a long time is not utilized; and secondly, the use of human expert knowledge may cause the embedding representations of the proteins to fall into local optima understood by humans, without further improving the representation capabilities of the proteins, thereby limiting the representation capabilities of the proteins.

### SUMMARY OF THE INVENTION

In view of the above, an object of the present invention is to provide a protein transformation method based on an amino acid knowledge graph and active learning. Representative proteins are selected through active learning in combination with knowledge in an amino acid knowledge graph, and the representative proteins and their manual annotations are used to assist in training of a protein property prediction model.

To achieve the above object of the invention, the present invention provides the following technical solution:

A protein transformation method based on an amino acid knowledge graph and active learning includes the following steps:
step 1: building an amino acid knowledge graph based on biochemical attributes of amino acids;
step 2: enhancing protein data in combination with the amino acid knowledge graph to obtain enhanced protein data, and performing representation learning to obtain first enhanced protein representations;
step 3: performing representation learning on the protein data or the protein data and the amino acid knowledge graph by using a pre-trained protein model to obtain second enhanced protein representations;
step 4: synthesizing the first enhanced protein representations and the second enhanced protein representations to obtain enhanced protein representations;
step 5: taking the enhanced protein representations as samples, and through active learning, screening out representative samples from the samples, manually annotating protein properties, and training a protein property prediction model by using the manually annotated representative samples; and
step 6: performing protein transformation by using the protein property prediction model.

Preferably, in the amino acid knowledge graph built in step 1, each triple is (an amino acid, a relationship, a biochemical attribute value), where the relationship is a relationship between the amino acid and the biochemical attribute value.

Preferably, in step 2, enhancing protein data in combination with the amino acid knowledge graph includes: for each amino acid in each piece of the protein data, finding a triple containing the amino acid from the amino acid knowledge graph, connecting the biochemical attribute corresponding to the amino acid in the triple as a new node into a protein structure, and taking a biochemical attribute value as an attribute value of the new node and the protein data connected to the biochemical attribute value as enhanced protein data.

Preferably, in step 2, representation learning is performed on the enhanced protein data by using a pluggable representation model to obtain the first enhanced protein representations, where the pluggable representation model includes a graph neural network model and a Transformer model.

Preferably, in step 3, during the process of performing representation learning on the protein data and the amino acid knowledge graph by using a pre-trained protein model, the representation learning is performed on triples (amino acids, relationships, biochemical attribute values) in the amino acid knowledge graph, as token-level additional information of the pre-trained protein model and the protein data as inputs to obtain the second enhanced protein representations.

Preferably, in step 4, the first enhanced protein representations and the second enhanced protein representations are synthesized by means of splicing, so as to obtain the enhanced protein representations.

Preferably, in step 5, during active learning, screening of the representative samples, manual annotation of the protein properties of the representative samples, and training of the protein property prediction model are performed for a plurality of rounds in an iterative cycle manner, where each round of iterative cycle includes:
(a): calculating Fisher Kernel distances between each unannotated sample in a sample space and all annotated samples, and selecting unannotated samples farthest away from all the annotated samples as the representative samples according to Fisher Kernel distance metrics; cyclically performing step (a) until k representative samples are obtained, and manually annotating the protein properties to obtain annotated samples; and in an initial round, taking samples closest to a midpoint in the sample space as initial annotated samples;
(b): training the protein property prediction model by using the k manually annotated representative samples screened out in the current round, and performing tag prediction on the unannotated samples by using the protein property prediction model trained in the current round to obtain prediction tags for the unannotated samples; and
(c): based on the Fisher Kernel distance metrics between the samples, screening out k1 samples with maximum Fisher Kernel distance metrics from the sample space, and updating a Fisher Kernel with a goal of making the annotated samples as dissimilar as possible and the unannotated samples as similar as possible in the k1 samples, where k1 is the number of current annotated samples present in the sample space.

Preferably, in step 6, performing protein transformation by using the protein property prediction model includes:
changing an amino acid sequence of original protein data to obtain a plurality of pieces of new protein data, and obtaining new enhanced protein representations corresponding to the new protein data by steps 2-4;
performing property prediction on the new enhanced protein representations by using the protein property prediction model to obtain predicted protein properties; and
selecting the new protein data as a transformed protein, where a difference between the predicted protein properties of the new protein data and the original protein properties corresponding to the original protein data is within a threshold range.

Compared with the prior art, the present invention has at least the following beneficial effects:

The amino acid knowledge graph is built based on various important biochemical attributes of the amino acids, and represents microscopic biochemical attribute relationships among the amino acids; the biochemical attributes of the amino acids, represented by the amino acid knowledge graph are connected to the protein data, to enhance the protein data, so that the enhanced protein representations corresponding to the enhanced protein data have both the data-driven protein semantic representation capability of the pre-trained protein model and the capability of protein attribute representation driven by knowledge of the amino acid knowledge graph;
the enhanced protein representations are taken as the samples, and through the active learning, the representative samples are screened out from the samples, the protein properties are manually annotated, and the protein property prediction model is trained by using the manually annotated representative samples, so that the protein property prediction model with the high prediction accuracy can be obtained with as few manual sample annotations as possible; and
moreover, the actively learnt samples are the enhanced protein representations containing both semantic representations and attribute representations, so that through active learning, semantic information contained in a huge unsupervised corpus can be utilized and the microscopic biochemical attribute relationships among the amino acids can be captured, thus enhancing the effect of active learning. In other words, the screened representative samples are more representative, have high quality, and are superior in training the protein property prediction model; and
the use of the protein property prediction model for protein transformation can achieve rapid and accurate protein transformation.

### BRIEF DESCRIPTION OF THE DRAWINGS

To more clearly illustrate the technical solutions in the embodiments of the present invention or in the prior art, the accompanying drawings that need to be used in the description of the embodiments or the prior art will be briefly described below. Apparently, the accompanying drawings in the description below merely illustrate some embodiments of the present invention. Those of ordinary skill in the art may also derive other accompanying drawings from these accompanying drawings without creative efforts.

FIG. 1 is a flowchart of a protein transformation method based on an amino acid knowledge graph and active learning provided by an embodiment;

FIG. 2 is a flowchart of training a protein property prediction model in combination with active learning provided by an embodiment; and

FIG. 3 is a schematic diagram of enhancing protein data in combination with an amino acid knowledge graph provided by an embodiment.

### DETAILED DESCRIPTION OF THE EMBODIMENTS

To make the object, the technical solutions and advantages of the present invention clearer, the present invention is further described in detail below in conjunction with the accompanying drawings and the embodiments. It should be understood that the specific embodiments described herein are merely used to explain the present invention, but not to limit the scope of protection of the present invention.

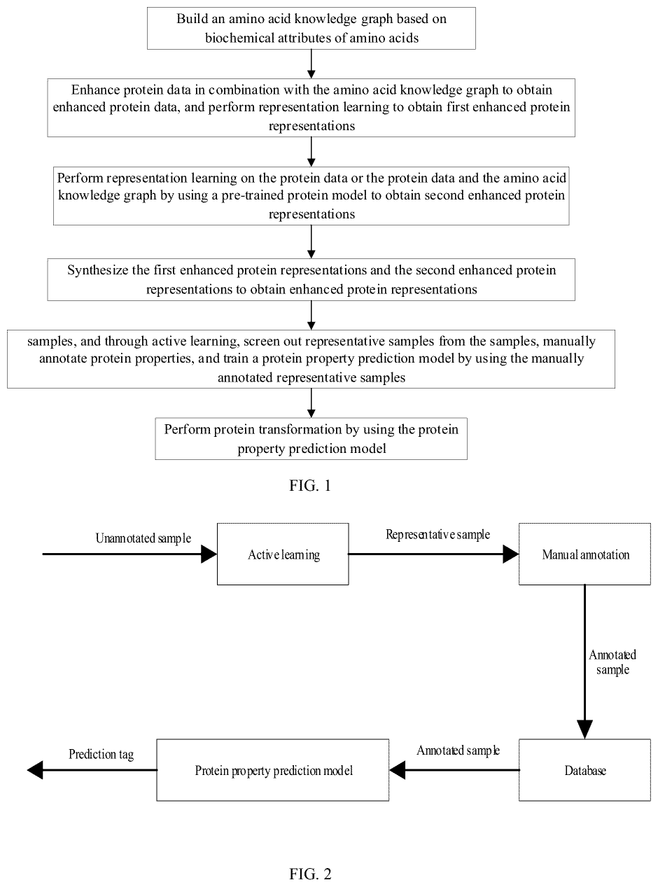

FIG. 1 is a flowchart of a protein transformation method based on an amino acid knowledge graph and active learning provided by a n embodiment. As shown in FIG. 1, a protein transformation method based on an amino acid knowledge graph and active learning provided by an embodiment includes steps below.

In step 1, an amino acid knowledge graph is built based on biochemical attributes of amino acids.

In this embodiment, the biochemical attributes of the amino acids refer to important biochemical properties selected by experts, generally collected from published papers, and specifically including a polarity, a hydrophilicity index, an aromatic or aliphatic series, a volume, an isoelectric point, a pKr value, a molecular weight, a dissociation constant (carboxyl), a dissociation constant (amino), and the flexibility, and the amino acid knowledge graph is built from the biochemical attribute information to obtain microscopic biochemical attribute relationships among various amino acids for subsequent encoding of protein embedding representations.

In the amino acid knowledge graph, the microscopic biochemical attribute relationships among the amino acids are represented in the form of triples, which are specifically represented as (amino acids, relationships, biochemical attribute values), where the relationships are relationships between the amino acids and the biochemical attribute values. For example, triples (Aromatic, isFamilyOf, Histidine) and (Arginine, hasPKrValue, 12.48) represent that the histidine belongs to the aromatic series, and the pKr value of the arginine is 12.48, respectively.

In step 2, protein data is enhanced in combination with the amino acid knowledge graph to obtain enhanced protein data, and representation learning is performed to obtain first enhanced protein representations.

The protein data refers to an amino acid sequence made up of several amino acids, and each piece of the protein data exhibits different protein properties due to the presence of a few special amino acids. The protein transformation is to change the special amino acids and/or positions, so as to obtain different protein properties.

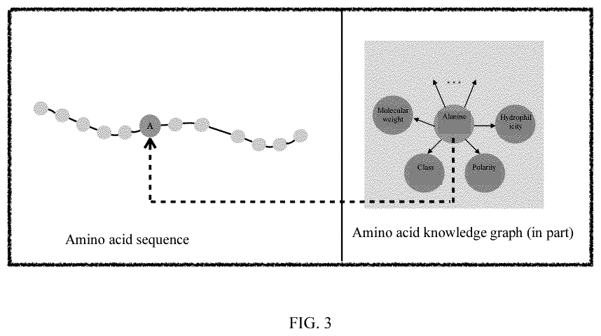

As shown in FIG. 3, in this embodiment, enhancing protein data in combination with the amino acid knowledge graph includes: for each amino acid in each piece of the protein data, finding a triple containing the amino acid from the amino acid knowledge graph, connecting the biochemical attribute corresponding to the amino acid in the triple as a new node into a protein structure, and taking a biochemical attribute value as an attribute value of the new node and the protein data connected to the biochemical attribute value as enhanced protein data.

After the enhanced protein data is obtained, the representation learning is performed on the enhanced protein data to obtain the first enhanced protein representations corresponding to the enhanced protein data. In this embodiment, the representation learning is performed on the enhanced protein data by using a pluggable representation model to obtain the first enhanced protein representations after knowledge enhancement, where the first enhanced protein representations contain both protein topological structures and biological protein domain knowledge. The pluggable representation model includes a graph neural network model and a transformer model. The first enhanced protein representations extracted using the graph neural network model (GNN model) not only include topological structure knowledge in the protein data, but also can capture microscopic relationships among amino acids that are not connected by peptide bonds.

In step 3, representation learning is performed on the protein data or the protein data and the amino acid knowledge graph by using a pre-trained protein model to obtain second enhanced protein representations.

The pre-trained protein model refers to a special model for extracting the protein embedding representations, and may be a pre-trained MSA-transformer model. The representation learning is performed on the protein data by using the pre-trained protein model such as the MSA-transformer model to obtain the second enhanced protein representations, where the second enhanced protein representations contain the topological structure knowledge in the protein data.

Certainly, the representation learning may also be performed on the protein data and the amino acid knowledge graph by using the pre-trained protein model, that is, the representation learning is performed on the triples (the amino acids, the relationships, the biochemical attribute values) in the amino acid knowledge graph, as token-level additional information of the pre-trained protein model and the protein data as inputs to obtain the second enhanced protein representations, where the second enhanced protein representations contain both the protein topological structures and the biological protein domain knowledge.

In step 4, the first enhanced protein representations and the second enhanced protein representations are synthesized to obtain enhanced protein representations.

In this embodiment, the first enhanced protein representations and the second enhanced protein representations are synthesized by means of splicing, so as to obtain the enhanced protein representations. The enhanced protein representation is expressed with the following formula:

x=concat(T(s),f(s,kg))

or

x=concat(T(s,kg),f(s,kg))
wherein s represents each piece of the protein data, that is, the amino acid sequence, kg represents information of the amino acid knowledge graph, f( ) represents the pluggable representation model, f (s, kg) represents a first enhanced protein representation obtained by learning enhanced protein data, obtained by adding kg to s, through the pluggable representation model, TO represents the pre-trained protein model, T(s) represents a second enhanced protein representation obtained by learning s through the pre-trained protein model, T (s, kg) represents a second enhanced protein representation obtained by learning enhanced protein data, obtained by adding kg to s, through the pre-trained protein model, concat( ) represents a splicing operation, and x represents the enhanced protein representation.

In this embodiment, the enhanced protein representation obtained through the splicing operation contains both potential semantic information of a large amount of unsupervised protein data and biological domain knowledge (prior knowledge of the biochemical attributes) contained in the amino acid expert knowledge graph in order to better represent a protein.

In step 5, the enhanced protein representations are taken as samples, and through active learning, representative samples are screened out from the samples, protein properties are manually annotated, and a protein property prediction model is trained by using the manually annotated representative samples.

In this embodiment, each enhanced protein representation is taken as a sample, all samples jointly constitute a sample space, active learning is performed in the sample space to screen out the representative samples for manual annotation, and the protein property prediction model is trained by using the manually annotated representative samples to efficiently improve the robustness of the protein property prediction model.

As shown in FIG. 2, during active learning, screening of the representative samples, manual annotation of the protein properties of the representative samples, and training of the protein property prediction model are performed for a plurality of rounds in an iterative cycle manner, wherein each round of iterative cycle includes steps below.

In step (a), Fisher Kernel distances between each unannotated sample in the sample space and all annotated samples are calculated, and the unannotated sample farthest away from all the annotated samples is selected as a representative sample according to Fisher Kernel distance metrics; and step (a) is cyclically performed until k representative samples are obtained, and the protein properties are manually annotated to obtain annotated samples.

It should be noted that in an initial round, the sample closest to a midpoint in the sample space is selected as an initial annotated sample, and then Fisher Kernel distances between all remaining samples in the sample space and the initial annotated sample are calculated, where k is a natural number set according to an application choice.

In this embodiment, the process that Fisher Kernel distances between each unannotated sample and all annotated samples are calculated includes steps below.

All first Fisher Kernel distances between each unannotated sample and all the annotated samples are calculated according to the enhanced protein representations corresponding to the samples, and a minimum is selected from all the first Fisher Kernel distances as a first Fisher Kernel distance for each unannotated sample, where the longer the first Fisher Kernel distance of the sample is, the larger the amount of information is, which is expressed with the following formula:

dₙₘ^{x}=∥xₙ−xₘ∥_{fk},m=1, . . . ,k;n=k+1, . . . ,N

dₙ^{x}=min(dₙₘ^{x}),m=1, . . . ,k;n=k+1, . . . ,N
wherein N is the total number of samples in the sample space, n is a serial number index of an unannotated sample, m represents a serial number index of an annotated sample, k is the number of annotated samples, ∥xₙ−xₘ∥_{fk} represents that a distance metric ∥xₙ−xₘ∥ is processed by a Fisher Kernel (fk) method, dₙₘ^{x} in represents a first Fisher Kernel distance between an n^{th} unannotated sample and an m^{th} annotated sample under a Fisher Kernel condition, min( ) represents the minimum, do represents a first Fisher Kernel distance for the n^{th} unannotated sample, and xₙ and xₘ represent enhanced protein representations of the n^{th} unannotated sample and the m^{th} annotated sample, respectively.

All second Fisher Kernel distances between each unannotated sample and all the annotated samples are calculated according to manual annotation tags for the annotated samples and the prediction tags for the unannotated samples, and a minimum is selected from all the second Fisher Kernel distances as a second Fisher Kernel distance for each unannotated sample, where the longer the second Fisher Kernel distance of the sample is, the larger the amount of information is, which is expressed with the following formula:

dₙₘ^{y}=∥yₙ−yₘ∥_{fk},m=1, . . . ,k;n=k+1, . . . ,N

dₙ^{y}=min(dₙₘ^{y}),m=1, . . . ,k;n=k+1, . . . ,N
wherein yₙ and yₘ represent a prediction tag for the n^{th} unannotated sample and a manual annotation tag for the m^{th} annotated sample, respectively, dₙₘ^{y} represents a second Fisher Kernel distance between the n^{th} unannotated sample and the n^{th} annotated sample under the Fisher Kernel condition, and dₙ^{y} represents a second Fisher Kernel distance for the n^{th} unannotated sample.

A Fisher Kernel distance for each unannotated sample includes the first Fisher Kernel distance dₙ^{x} and the second Fisher Kernel distance dₙ^{y}.

In this embodiment, the unannotated sample farthest away from all the annotated samples may be selected as the representative sample according to the Fisher Kernel distance metrics in two ways below.

In the first way, fusion is performed on the first Fisher Kernel distance and the second Fisher Kernel distance for each unannotated sample to obtain a first fused Fisher Kernel distance for each unannotated sample, and the unannotated sample corresponding to a maximum first fused Fisher Kernel distance is selected as the representative sample, which is expressed with the following formula:

dₙ^{xy}=F(dₙ^{x},dₙ^{y}),n=k+1, . . . ,N
where F( ) represents the fusion operation, and dₙ^{xy} represents the first fused Fisher Kernel distance.

In the second way, fusion is performed on the first Fisher Kernel distance and the second Fisher Kernel distance for each unannotated sample relative to each annotated sample, to obtain a second fused Fisher Kernel distance for each unannotated sample relative to each annotated sample, and the unannotated sample corresponding to a maximum second fused Fisher Kernel distance is selected as the representative sample, which is expressed with the following formula:

dₙ^{xy,}=F(dₙₘ^{x},dₙₘ^{y}),m=1, . . . ,k;n=k+1, . . . ,N
wherein dₙ^{xy,} represents the second fused Fisher Kernel distance.

In step (b), the protein property prediction model is trained by using the k manually annotated representative samples screened out in the current round, and tag prediction is performed on the unannotated samples by using the protein property prediction model trained in the current round to obtain prediction tags for the unannotated samples.

In this embodiment, the k representative samples screened out in each round are used for training the protein property prediction model after being manually annotated; after each round of training, the tag prediction is performed on the unannotated samples by using the trained protein property prediction model to obtain the prediction tags for the unannotated samples, where the prediction tags are further used for building a loss function for active learning in the current round to update Fisher Kernel parameters for active learning.

In this embodiment, the protein property prediction model is used to predict the protein properties, and the protein properties may be predicted by using a multiple and pluggable regression model, using a custom model architecture, or directly calling an encapsulated regression model in keras, sklearn, and xgboost.

In step (c), based on the Fisher Kernel distance metrics between the samples, k1 samples with maximum Fisher Kernel distance metrics are screened out from the sample space, and a Fisher Kernel is updated with a goal of making the annotated samples as dissimilar as possible and the unannotated samples as similar as possible in the k1 samples, where k1 is the number of current annotated samples present in the sample space.

In step (c), the calculation of the Fisher Kernel distances between the samples is basically the same as the calculation of the Fisher Kernel distances between each unannotated sample and all the annotated samples, and there is a difference that calculation objects for the Fisher Kernel distances are different, so that it is not confirmed whether the samples are annotated in step (c). Specifically, there being based on the Fisher Kernel distance metrics between the samples includes steps below.

All Fisher Kernel distances between each sample and all other samples are calculated according to the enhanced protein representations corresponding to the samples, and a minimum is selected from all the Fisher Kernel distances as a first Fisher Kernel distance for each sample, where the longer the first Fisher Kernel distance of the sample is, the larger the amount of information is, and the samples include the annotated samples and the unannotated samples, which is expressed with the following formula:

dᵢⱼ^{x}=∥xᵢ−xⱼ∥_{fk},i≠j,i=1,2, . . . ,N,j=1,2, . . . ,N

dᵢ^{x}=min(dᵢⱼ^{x})
wherein N is the total number of samples in the sample space, both i and j are serial number indexes of the samples, ∥xᵢ−xⱼ∥_{fk} represents that a distance metric ∥xᵢ−xⱼ∥ is processed by a Fisher Kernel (fk) method, dᵢⱼ^{x} represents a first Fisher Kernel distance between an i^{th} sample and a j^{th} sample under a Fisher Kernel condition, min( ) represents the minimum, dᵢ^{x} represents a first Fisher Kernel distance for the i^{th} sample, and xᵢ and xⱼ represent enhanced protein representations of the i^{th} sample and the i^{th} sample, respectively.

All Fisher Kernel distances between each sample and all other samples are calculated according to manual annotation tags for the annotated samples and/or the prediction tags for the unannotated samples, and a minimum is selected from all the Fisher Kernel distances as a second Fisher Kernel distance for each unannotated sample, which is expressed with the following formula:

dᵢⱼ^{y}=∥yᵢ−yⱼ∥_{fk},i≠j,i=1,2, . . . ,N,j=1,2, . . . ,N

dᵢ^{y}=min(dᵢⱼ^{y})
wherein yᵢ and yⱼ represent a tag (a manual annotation tag or a prediction tag) for the i^{th} sample, and a tag (a manual annotation tag or a prediction tag) for the jₜₕ sample, respectively, d_{iy}^{j} represents a second Fisher Kernel distance between the i^{th} sample and the j^{th} sample under the Fisher Kernel condition, and dᵢ^{y} represents a second Fisher Kernel distance for the i^{th} sample.

A Fisher Kernel distance for each sample includes the first Fisher Kernel distance dᵢ^{x} and the second Fisher Kernel distance dᵢ^{y}.

Based on this, in this embodiment, the k1 samples with the maximum Fisher Kernel distance metrics may be screened out from the sample space in two ways below.

In the first way, fusion is performed on the first Fisher Kernel distance and the second Fisher Kernel distance for each sample to obtain a first fused Fisher Kernel distance for each sample, and first k1 large samples corresponding to the first fused Fisher Kernel distances are selected as k1 samples obtained by screening.

In the second way, fusion is performed on the first Fisher Kernel distance and the second Fisher Kernel distance for each sample relative to each other sample, to obtain a second fused Fisher Kernel distance for each sample relative to each other sample, and first k1 large unannotated samples corresponding to the second fused Fisher Kernel distances are selected as k1 samples obtained by screening.

In this embodiment, metric learning methods of the Fisher Kernel are combined, vectorized data may be better distinguished in a certain extent by using these methods, and the Fisher Kernel is continuously updated in the model. The use of different learnable Fisher kernels for calculation makes the differentiation of the Fisher Kernel distance metrics more sensitive to different samples. The expert knowledge contained in the protein knowledge graph and the potential semantic knowledge represented by the pre-trained protein model are combined to assist in active learning, so that the samples selected in this way are more representative. These representative samples are manually annotated to train the protein property prediction model, which can improve the training efficiency and reduce the time and cost of annotation.

In step 6, protein transformation is performed by using the protein property prediction model.

In this embodiment, the process that protein transformation is performed by using the protein property prediction model includes steps below.

An amino acid sequence of original protein data is changed to obtain a plurality of pieces of new protein data, and new enhanced protein representations corresponding to the new protein data are obtained by steps 2-4.

Property prediction is performed on the new enhanced protein representations by using the protein property prediction model to obtain predicted protein properties.

The new protein data is selected as a transformed protein, where a difference between the predicted protein properties of the new protein data and the original protein properties corresponding to the original protein data is within a threshold range.

In this embodiment, the threshold range is customized according to application requirements, and when the amino acid sequence of the original protein data is changed, special amino acids that play a role in protein properties are generally selected for replacement of original positions or adjustment of amino acid positions. For obtaining a plurality of pieces of new protein data, due to the powerful computing power of the protein property prediction model, property prediction is simultaneously performed on all the new protein data, and transformable protein structures are screened out according to prediction results.

The specific implementation mentioned above provides a detailed description of the technical solution and beneficial effects of the present invention. It should be understood that the above is only the optimal embodiment of the present invention and is not intended to limit the present invention. Any modifications, supplements, and equivalent substitutions made within the scope of principle of the present invention should be included in the scope of protection of the present invention.

1. A protein transformation method based on an amino acid knowledge graph and active learning, comprising the following steps:
step 1: building an amino acid knowledge graph based on biochemical attributes of amino acids;
step 2: enhancing protein data in combination with the amino acid knowledge graph to obtain enhanced protein data, and performing representation learning to obtain first enhanced protein representations;
step 3: performing representation learning on the protein data or the protein data and the amino acid knowledge graph by using a pre-trained protein model to obtain second enhanced protein representations;
step 4: synthesizing the first enhanced protein representations and the second enhanced protein representations to obtain enhanced protein representations;
step 5: taking the enhanced protein representations as samples, and through active learning, screening out representative samples from the samples, manually annotating protein properties, and training a protein property prediction model by using the manually annotated representative samples; and
step 6: performing protein transformation by using the protein property prediction model.

2. The protein transformation method based on an amino acid knowledge graph and active learning according to claim 1, wherein in the amino acid knowledge graph built in the step 1, each triple includes an amino acid, a relationship, and a biochemical attribute value, wherein the relationship is a relationship between the amino acid and the biochemical attribute value.

3. The protein transformation method based on an amino acid knowledge graph and active learning according to claim 1, wherein in the step 2, enhancing protein data in combination with the amino acid knowledge graph comprises: for each amino acid in each piece of the protein data, finding a triple containing the amino acid from the amino acid knowledge graph, connecting the biochemical attribute corresponding to the amino acid in the triple as a new node into a protein structure, and taking a biochemical attribute value as an attribute value of the new node and the protein data connected to the biochemical attribute value as enhanced protein data.

4. The protein transformation method based on an amino acid knowledge graph and active learning according to claim 1, wherein in the step 2, representation learning is performed on the enhanced protein data by using a pluggable representation model to obtain the first enhanced protein representations, wherein the pluggable representation model comprises a graph neural network model and a Transformer model.

5. The protein transformation method based on an amino acid knowledge graph and active learning according to claim 1, wherein in the step 3, during the process of performing representation learning on the protein data and the amino acid knowledge graph by using a pre-trained protein model, the representation learning is performed on triples including amino acids, relationships, biochemical attribute values in the amino acid knowledge graph, as token-level additional information of the pre-trained protein model and the protein data as inputs to obtain the second enhanced protein representations.

6. The protein transformation method based on an amino acid knowledge graph and active learning according to claim 1, wherein in the step 4, the first enhanced protein representations and the second enhanced protein representations are synthesized by means of splicing, so as to obtain the enhanced protein representations.

7. The protein transformation method based on an amino acid knowledge graph and active learning according to claim 1, wherein in the step 5, during active learning, screening of the representative samples, manual annotation of the protein properties of the representative samples, and training of the protein property prediction model are performed for a plurality of rounds in an iterative cycle manner, wherein each round of iterative cycle comprises:
(a): calculating Fisher Kernel distances between each unannotated sample in a sample space and all annotated samples, and selecting the unannotated sample farthest away from all the annotated samples as a representative sample according to Fisher Kernel distance metrics; cyclically performing step (a) until k representative samples are obtained, and manually annotating the protein properties to obtain annotated samples; and in an initial round, taking the sample closest to a midpoint in the sample space as an initial annotated sample;
(b): training the protein property prediction model by using the k manually annotated representative samples screened out in the current round, and performing tag prediction on the unannotated samples by using the protein property prediction model trained in the current round to obtain prediction tags for the unannotated samples; and
(c): based on the Fisher Kernel distance metrics between the samples, screening out k1 samples with maximum Fisher Kernel distance metrics from the sample space, and updating a Fisher Kernel with a goal of making the annotated samples as dissimilar as possible and the unannotated samples as similar as possible in the k1 samples, wherein k1 is the number of current annotated samples present in the sample space.

8. The protein transformation method based on an amino acid knowledge graph and active learning according to claim 7, wherein in the step (a), calculating Fisher Kernel distances between each unannotated sample and all annotated samples comprises:
calculating all first Fisher Kernel distances between each unannotated sample and all the annotated samples according to the enhanced protein representations corresponding to the samples, and selecting a minimum from all the first Fisher Kernel distances as a first Fisher Kernel distance for each unannotated sample, wherein the longer the first Fisher Kernel distance of the sample is, the larger the amount of information is; and
calculating all second Fisher Kernel distances between each unannotated sample and all the annotated samples according to manual annotation tags for the annotated samples and the prediction tags for the unannotated samples, and selecting a minimum from all the second Fisher Kernel distances as a second Fisher Kernel distance for each unannotated sample, wherein the longer the second Fisher Kernel distance of the sample is, the larger the amount of information is;
wherein a Fisher Kernel distance for each unannotated sample comprises the first Fisher Kernel distance and the second Fisher Kernel distance;
in the step (a), selecting the unannotated sample farthest away from all the annotated samples as a representative sample according to Fisher Kernel distance metrics comprises:
performing fusion on the first Fisher Kernel distance and the second Fisher Kernel distance for each unannotated sample to obtain a first fused Fisher Kernel distance for each unannotated sample, and selecting the unannotated sample corresponding to a maximum first fused Fisher Kernel distance as the representative sample;
or performing fusion on the first Fisher Kernel distance and the second Fisher Kernel distance for each unannotated sample relative to each annotated sample, to obtain a second fused Fisher Kernel distance for each unannotated sample relative to each annotated sample, and selecting the unannotated sample corresponding to a maximum second fused Fisher Kernel distance as the representative sample.

9. The protein transformation method based on an amino acid knowledge graph and active learning according to claim 7, wherein in the step (c), there being based on the Fisher Kernel distance metrics between the samples comprises:
calculating all Fisher Kernel distances between each sample and all other samples according to the enhanced protein representations corresponding to the samples, and selecting a minimum from all the Fisher Kernel distances as a first Fisher Kernel distance for each sample, wherein the longer the first Fisher Kernel distance of the sample is, the larger the amount of information is, and the samples comprise the annotated samples and the unannotated samples; and
calculating all Fisher Kernel distances between each sample and all other samples according to manual annotation tags for the annotated samples and the prediction tags for the unannotated samples, and selecting a minimum from all the Fisher Kernel distances as a second Fisher Kernel distance for each unannotated sample, wherein the longer the second Fisher Kernel distance of the sample is, the larger the amount of information is;
wherein a Fisher Kernel distance for each sample comprises the first Fisher Kernel distance and the second Fisher Kernel distance;
in the step (c), screening out k1 samples with maximum Fisher Kernel distance metrics from the sample space comprises:
performing fusion on the first Fisher Kernel distance and the second Fisher Kernel distance for each sample to obtain a first fused Fisher Kernel distance for each sample, and selecting first k1 large samples corresponding to the first fused Fisher Kernel distances as k1 samples obtained by screening;
or performing fusion on the first Fisher Kernel distance and the second Fisher Kernel distance for each sample relative to each other sample, to obtain a second fused Fisher Kernel distance for each sample relative to each other sample, and selecting first k1 large unannotated samples corresponding to the second fused Fisher Kernel distances as k1 samples obtained by screening.

10. The protein transformation method based on an amino acid knowledge graph and active learning according to claim 1, wherein in the step 6, performing protein transformation by using the protein property prediction model comprises:
changing an amino acid sequence of original protein data to obtain a plurality of pieces of new protein data, and obtaining new enhanced protein representations corresponding to the new protein data by the steps 2-4;
performing property prediction on the new enhanced protein representations by using the protein property prediction model to obtain predicted protein properties; and
selecting the new protein data as a transformed protein, wherein a difference between the predicted protein properties of the new protein data and the original protein properties corresponding to the original protein data is within a threshold range.
